(19) United States
(12) Patent Application Publication
(10) Pub. No.: US 2010/0138959 A1
(43) Pub. Date: Jun. 3, 2010

(54) Method of Fabricating Bioinsecticide Obtained from Mammalian Galectin

(75) Inventors: Rong-Nan Huang, Taipei (TW); Nein-Ting Chen, Danshui Town (TW); Shiu-Hwa Wang, Xindian City (TW); Ling-Long Huang, Taipei (TW); Shiang-Jiuun Chen, Taipei (TW); Shaw-Jye Wu, Jhongli City (TW)

Correspondence Address: Jackson Intellectual Property Group PLLC, 106 Starvale Lane, Shipman, VA, 22971, US

(73) Assignee: National Taiwan University, Taipei (TW)

(21) Appl. No.: 12/629,512

(22) Filed: Dec. 2, 2009

(30) Foreign Application Priority Data
Dec. 3, 2008 (TW) 097146900

(51) Int. Cl. A01H 1/00

(52) U.S. Cl. 800/279

(57) ABSTRACT

A bioinsecticide is fabricated. Galectin widely found in mammalian is used for the fabrication. The bioinsecticide thus fabricated targets on chitin of peritrophic membrane in intestinal tract for killing insect. The bioinsecticide can be easily decomposed. The present invention is highly safe; is environment protected; and can be widely applied.

### FIELD OF THE INVENTION

The present invention relates to fabricating a bioinsecticide; more particularly, relates to using mammalian galectin for fabricating a bioinsecticide targeting on chitin of peritrophic membrane to kill insect with high safety, easy decomposition, environment protection and wide application.

### DESCRIPTION OF THE RELATED ART

Following the development of human society, environment protection becomes crucial. Although pesticide has big contribution to provisions, overuse of it is a major cause of pollution in the environment and food chain. Main pesticide is insecticide, where there are more than 200 kinds of insecticide. Overuse of pesticide makes cost higher and may cause harm to human health. The use of pesticide may even make insect resistant to the specific pesticide, which may result in heavier dose of the pesticide used or make the pesticide useless. In the end, an ecological crisis may come if things just let go.

Therefore, some studies are worked on for minimizing the impact of pesticide on environment, where pesticide safe to human and livestock and environment protected are trying to be developed. An achievement is to use bio-pesticide to replace chemical pesticide having high toxicity, high residual and high resistance. For example, a gene of a germ may be added into a plant through a transgenic method for obtaining toxin. And insect eating the plant may thus be killed. However, resistance may still be found in the insect on overusing. Hence, the prior art does not fulfill all users' requests on actual use.

### SUMMARY OF THE INVENTION

The main purpose of the present invention is to use mammalian galectin to fabricate a bioinsecticide targeting on chitin of peritrophic membrane to kill insect with high safety, easy decomposition, environment protection and wide application

To achieve the above purpose, the present invention is a method of fabricating a bioinsecticide obtained from a mammalian galectin, comprising steps of: providing a galectin gene from a mammalian which targets on chitin of peritrophic membrane; and, transferring the galectin gene directly onto a plant to obtain a resistant plant. Accordingly, a novel method of fabricating a bioinsecticide obtained from a mammalian galectin is obtained.

### BRIEF DESCRIPTIONS OF THE DRAWINGS

The present invention will be better understood from the following detailed description of the preferred embodiment according to the present invention, taken in conjunction with the accompanying drawings, in which

FIG. 1 is the flow view showing the first preferred embodiment according to the present invention;

FIG. 2 is the flow view showing the second preferred embodiment;

FIG. 3 is the view showing the toxicity of GAL1 protein effecting Plutella xylostella;

FIG. 4 is the view showing the GAL1 protein in the Plutella xylostella;

FIG. 5A is the view showing the recombinant GAL1 protein purified by E. coli;

FIG. 5B is the view showing the GAL1 protein in the arabidopsis plant; and

FIG. 6A to FIG. 6C are the views showing the survival rates, the weight expressions and the leaf consumptions under the toxicity.

### DESCRIPTIONS OF THE PREFERRED EMBODIMENTS

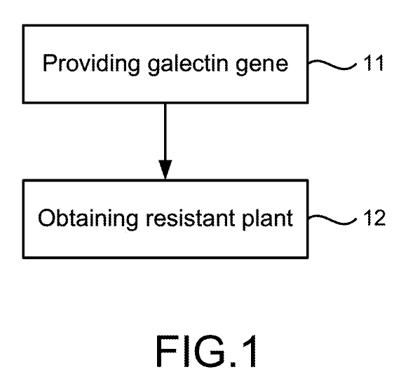

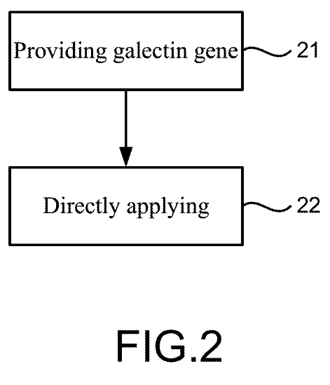

Please refer to FIG. 1 and FIG. 2, which are flow views showing a first and a second preferred embodiments according to the present invention. As shown in the figures, the present invention is a method of fabricating a bioinsecticide obtained from a mammalian galectin, where a mammalian galectin is used for fabricating a bioinsecticide targeting on chitin of peritrophic membrane. In a first preferred embodiment, the present invention comprises the following steps:

(a1) Providing galectin gene 11: A galectin gene is provided for fabricating a bioinsecticide, where the galectin gene is obtained from a mammalian and the bioinsecticide targets on chitin of peritrophic membrane; and

(b1) Obtaining resistant plant 12: The galectin gene is directly transferred onto a plant to obtain a resistant plant.

In a second preferred embodiment, the present invention comprises the following steps:

(a1) Providing galectin gene 11: A galectin gene is provided for fabricating a bioinsecticide, where the galectin gene is obtained from a mammalian and the bioinsecticide targets on chitin of peritrophic membrane; and

(b2) Directly applying 12: The galectin gene is constructed on a microbial plant to be mass-produced through a fermentation technology. Then the galectin gene is directly applied on a plant after purifying recombinant protein.

On using the present invention, a GAL1 protein is obtained from an ovarian cell of a hamster as a target. The GAL1 protein is constructed on a pQE31 carrier to be cultured with an E. coli SG13009-pQE31-GAL1 liquid for 12 to 16 hours (hr). A millimole of isopropyl-β-D-thiogalatopyranoside (IPTG) is added to be processed for 4 hrs to induce expression of GAL1 protein. Then the germ in the liquid is broken with Franch Press for collecting cell lysate in a centrifuge tube. A supernatant liquid is collected after being rotated with a centrifugal force of 10000 grams (g) at 4 Celsius degrees (° C.) for 30 minutes (min). An ammonium sulfate having 70% saturation is added for salting-out and settling protein followed with 10 min of stirring. A rotation is then processed with a centrifugal force of 15000 g at 4° C. for 30 min and a dialysis is processed with a lysis buffer at 4° C. overnight. After washing and eluting the germ extract through a Ni-NTA tube, an effluent is collected and dialyzed for obtaining 10 micrograms (μg) of protein to be separated through sodium godecyl sulfate polyacrylamide gel electrophoresis (SDS-PAGE). Thus, a recombinant GAL1 protein having histidine tagged is purified out.

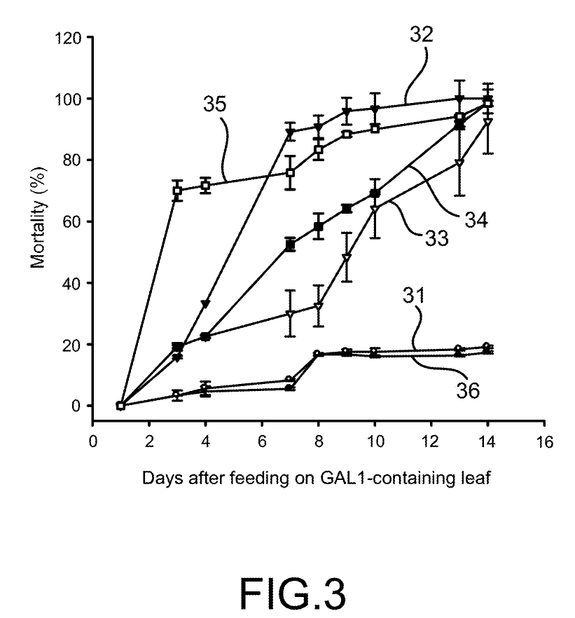

Please refer to FIG. 3, which is a view showing toxicity of GAL1 protein effecting Plutella xylostella. As shown in the figure, cabbages each having a circular diameter of 3 centimeters are separately sunk in a SG13009 germ solution, a GAL1 exact solution and GAL1 solutions having concentrations of 0.1 mg/ml, 0.2 mg/ml and 0.4 mg/ml separately for 30 seconds. After being fully dried and put in culture dishes, 3-year-old larvae of Plutella xylostella are cultivated at 25° C. in each dish and their survival rate is recorded each day. A trend curve 31 for the larvae in the SG13009 germ solution, a trend curve 32 for the larvae in the GAL1 exact solution, a trend curve 33 for the larvae in the GAL1 solution having 0.1 mg/ml concentration, a trend curve 34 for the larvae in the GAL1 solution having 0.2 mg/ml concentration and a trend curve 35 for the larvae in the GAL1 solution having 0.4 mg/ml concentration are compared to a trend curve 36 for the larvae in a control set. As results show, toxicity of the GAL1 protein is related to mortality of Plutella xylostella larva, where a higher toxicity results in a higher mortality.

For knowing effect of the GAL1 protein toxicity to intestinal tissue of Plutella xylostella, Plutella xylostella larvae are fed with cabbages sunk in GAL1 solutions having different concentrations for 48 and 96 hrs. After washing, dehydrating, staining, infiltrating and embedding with FPGA as a fixing agent, paraffin sections of intestinal tissues of Plutella xylostella are viewed. The larvae in the control set, which eat cabbage sunk in a solution having no GAL1 protein, have intact intestinal tissues and peritrophic membranes are formed, while other larvae eating cabbages sunk in solutions having GAL1 protein do not have intact peritrophic membranes formed but germs distributed on intestinal walls. Hence, it is known that Plutella xylostella larva eating cabbage sunk in a solution having a higher GAL1 protein concentration has their intestinal epithelial cells more seriously damaged; and insecticidal effect of GAL1 is proved to be related to the damage of the intestinal tissue.

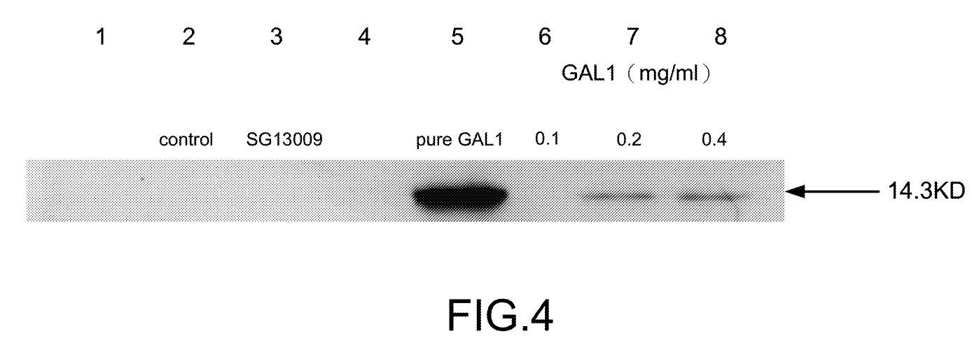

Please refer to FIG. 4, which is a view showing GAL1 protein in Plutella xylostella. As shown in the figure, Plutella xylostella larvae eating cabbages sunk in GAL1 solutions having different concentrations for 48 hrs are collected. A few larvae are picked up from each GAL1 solution to be put into a centrifuge tube. An extraction buffer is added to be squeezed up and down by a grinding rod. After being homogenized, a rotation is processed with a centrifugal force of 12000 g at 4° C. for 10 min to precipitate unbroken tissue. A supernatant liquid is thus obtained as a protein extract to detect GAL1 protein through Western blotting with a pure GAL1 protein as a positive control. As result shows, GAL1 protein is found in Plutella xylostella.

In addition, after feeding 3- to 4-year-old larvae of Plutella xylostella with cabbages having GAL1 protein for hrs or feeding with cabbages having 0.4 mg/ml GAL1 protein for different hours, GAL1 protein in the intestinal tissue is viewed for its distribution by using a monoclonal antibody of GAL1 protein and a secondary antibody linked with Flourescein Isothiocyanate (FITC). As results show, it is found that the distribution of GAL1 protein in the intestinal tissue is denser after higher concentration of GAL1 protein and longer processing time.

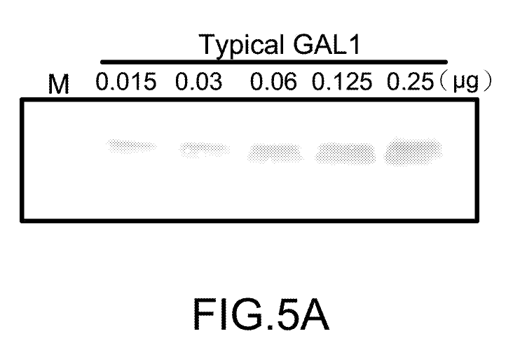

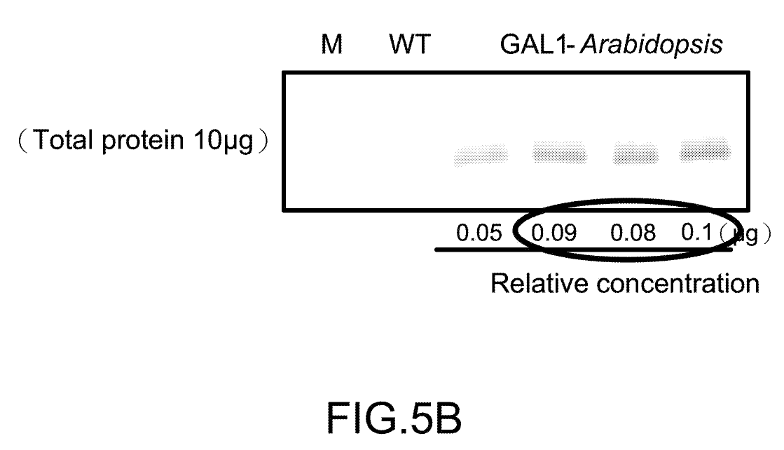

Please refer to FIG. 5A and FIG. 5B, which are a view showing recombinant GAL1 protein purified by E. coli; and a view showing GAL1 protein in an arabidopsis plant. As shown in the figures, GAL1 genes are directly constructed on a carrier of pCAMBIA1390-SUC2pro (for vascular bundle sieve tube expression) and a carrier of pCAMBIA1390-S35 (for whole plant expression); and then are transferred to an arabidopsis plant for expression. After screening for three generations, a GAL1 homozygous plant is obtained; and 10 μg of protein of the arabidopsis plant is obtained for SDS-PAGE separation. Then a monoclonal antibody of GAL1 is used for western blotting. In FIG. 5B, the arabidopsis plant transferred with GAL1 has an expression of GAL1 protein about 0.05˜0.1% of whole protein.

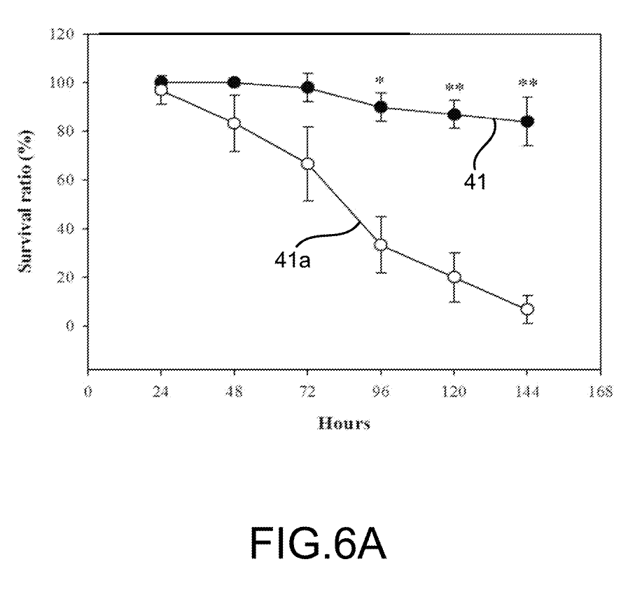

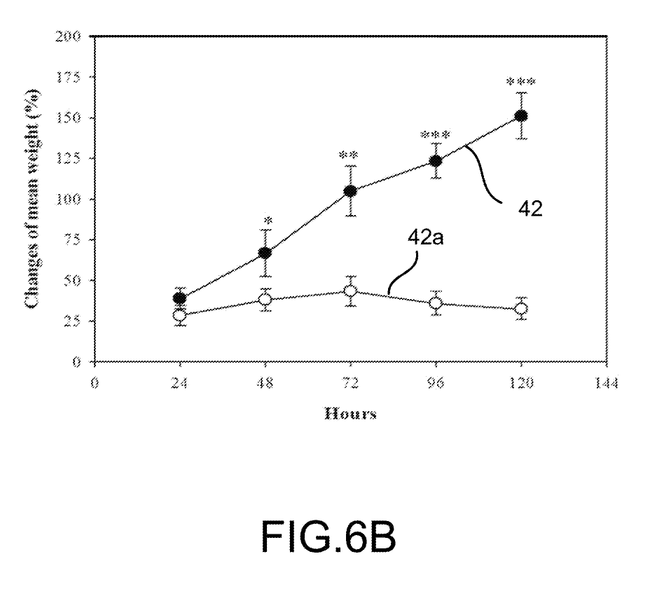

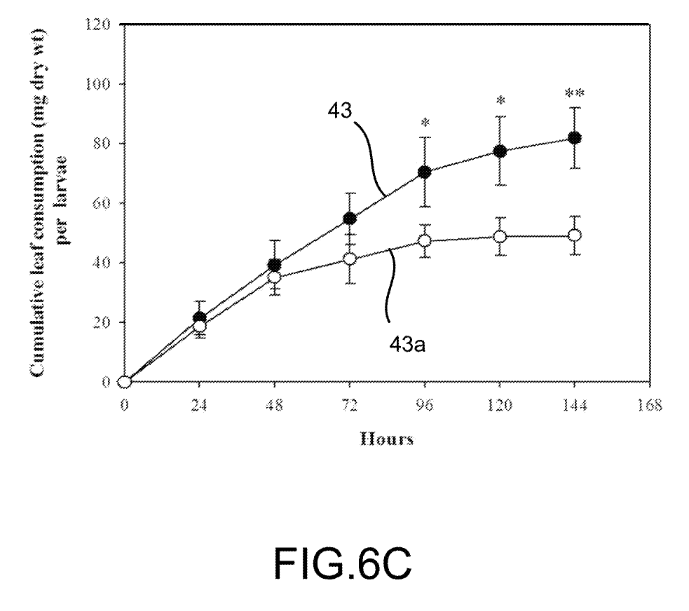

Please refer to FIG. 6A to FIG. 6C, which are views showing survival rates, weight expressions and leaf consumptions under toxicity. As shown in the figures, 3-year-old larvae of Plutella xylostella fed with wild type arabidopsis are obtained as control set for contrasting with 3-year-old larvae of Plutella xylostella fed with arabidopsis plant having GAL1 transferred for various periods of time. As results show, on comparing to a first survival rate expression curve 41, a first weight expression curve 42 and a first leaf consumption expression curve 43 of the Plutella xylostella larvae of the control set, a second survival rate expression curve 41a, a second weight expression curve 42a and a second leaf consumption expression curve 43a of the Plutella xylostella larvae fed with GAL1 transferred arabidopsis plant are obviously sloped down. It is thus proved that the GAL1 transferred arabidopsis plant has obvious toxicity to Plutella xylostella.

Then, peritrophic membrane is obtained from intestinal tract of Plutella xylostella through anatomy. After being directly reacted with GAL1 protein at 4° C. overnight, the peritrophic membrane is sequentially processed through secondary antibody reactions with GAL1 monoclonal antibody and FITC and is viewed with a confocal fluorescence microscope. A sample of actin and antibody is obtained as a negative set and a combination of a fluorescent whitening agent and chitin is obtained as a positive set for staining and viewing. As result shows, only GAL1 protein is uniquely fixed with the peritrophic membrane of the intestinal tract of Plutella xylostella.

In addition, peritrophic membranes of Plutella xylostella larvae fed with GAL1 transferred arabidopsis plants for hrs, 47 hrs and 72 hrs separately are obtained and a peritrophic membrane of a Plutella xylostella larva fed with wild type arabidopsis plant is obtained as a control set, where their microstructures are viewed with an electron microscope. It is found that the peritrophic membrane of the Plutella xylostella larvae fed with GAL1 transferred arabidopsis plants are obviously damaged; and the longer the larvae are fed, the damages are more serious. It is thus proved that GAL1 protein has a positive relationship to time for the fixation to the peritrophic membrane of the intestinal tract of Plutella xylostella.

GAL1 protein solution and bovine serum albumin (BSA, as a negative set) are obtained to be mixed with chitin powder. After being reacted at 4° C. overnight, a centrifugal rotation is processed to obtain chitin for SDS-PAGE separation. As results show, only GAL1 protein has a co-precipitation with chitin, while BSA doesn't. It is thus concluded that toxicity of the GAL1 transferred arabidopsis plant to Plutella xylostella is originated from the fixation of the GAL1 protein to chitin in the peritrophic membrane of Plutella xylostella; and the peritrophic membrane is thus damaged to cause death of the larvae.

As stated above, GAL1 protein has toxicity for killing insect. The present invention uses galectin GAL1 gene for being transferred to a plant as a bioinsecticide for killing insect. Or, the present invention mass-produces GAL1 protein through a fermentation technology to be directly used as bioinsecticide.

The present invention uses galectin, a basic gene found in mammalian, for fabricating bioinsecticide targeting on chitin to damage peritrophic membrane in intestinal tract. In this way, the present invention obtains the following advantages:

(a) Galectin is obtained from mammalian itself and so has no toxicity and the bioinsecticide thus made is highly safe.

(b) The target for galectin is chitin, which is widely existed in peritrophic membrane of bone and intestinal tract of insect; and thus the present invention can be widely applied.

(c) Galectin is a protein, so it can be easily decomposed at a high temperature and is environment protected.

(d) There are 14 galectin genes in mammalian; and the present invention is thus has a great potential for fabricating useful bioinsecticide.

To sum up, the present invention is a method of fabricating a bioinsecticide obtained from a mammalian galectin, where galectin is used for fabricating bioinsecticide with high safety, easy decomposition, environment protection and wide application.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of fabricating a bioinsecticide obtained from a mammalian galectin, comprising steps of:
(a1) providing a galectin gene, said galectin gene being obtained from a mammalian, said galectin gene targeting on chitin of peritrophic membrane; and
(b1) transferring said galectin gene directly onto a plant to obtain a resistant plant.

2. A method of fabricating a bioinsecticide obtained from a mammalian galectin, comprising steps of:
(a2) providing a galectin gene, said galectin gene being obtained from a mammalian, said galectin gene targeting on chitin of peritrophic membrane; and
(b2) constructing said galectin gene on a microbial plant to be mass-produced through a fermentation technology and directly applied on a plant after purifying recombinant protein.
